(19) United States
(12) Patent Application Publication
(10) Pub. No.: US 2024/0157122 A1
(43) Pub. Date: May 16, 2024

(54) HUMAN BODY ENHANCED TRAINING SYSTEM

(71) Applicant: Techfit Group LLC, Minneapolis, MN (US)

(72) Inventor: Jim Stathis, Minneapolis, MN (US)

(21) Appl. No.: 18/198,319

(22) Filed: May 17, 2023

Related U.S. Application Data
(63) Continuation of application No. 17/175,739, filed on Feb. 15, 2021, now abandoned.
(60) Provisional application No. 62/976,108, filed on Feb. 13, 2020.

(51) Int. Cl. A61N 1/04; A61N 1/36; A62B 17/00

(52) U.S. Cl. CPC A61N 1/0484; A61N 1/0452; A61N 1/0476; A61N 1/36003; A61N 1/36034; A62B 17/003; A61F 2007/0233

(57) ABSTRACT

A human body enhanced training system including a smart garment, a control module, a plurality of electrodes and at least one sensor. The smart garment is suitable for being worn by a person. When the smart garment is worn by the person, the smart garment covers at least a portion of the person's body. The control module is mounted with respect to the smart garment. The plurality of electrodes is operably connected to the control module. The plurality of electrodes is capable of stimulating the person's muscles. The at least one sensor is operably connected to the control module. The at least one sensor is capable of collecting physiological data.

### REFERENCE TO RELATED APPLICATION

This application is a continuation of U.S. application Ser. No. 17/175,739 filed on Feb. 15, 2021, which claims priority to Provisional Applic. No. 62/976,108, filed on Feb. 13, 2020, the contents of which are incorporated herein by reference.

### FIELD OF THE INVENTION

The invention relates generally to training system. More particularly, the invention relates to a human body enhanced training system.

### BACKGROUND OF THE INVENTION

It is recognized that electrical stimulation of muscles provides benefits such as enhancing the ability of the muscles to recover such as from exercise. However, the ability to control the use of electrical muscle stimulation and maximize the associated benefits has heretofore been limited.

### SUMMARY OF THE INVENTION

An embodiment of the invention is directed to a human body enhanced training system that includes a smart garment, a control module, a plurality of electrodes and at least one sensor. The smart garment is suitable for being worn by a person. When the smart garment is worn by the person, the smart garment covers at least a portion of the person's body. A control module is mounted with respect to the smart garment. A plurality of electrodes operably is connected to the control module. The plurality of electrodes is capable of stimulating the person's muscles. The at least one sensor is operably connected to the control module. The at least one sensor is capable of collecting physiological data.

Another embodiment of the invention is directed to a method of training. A smart garment is provided that is suitable for being worn by a person. The person has a body and muscles. The smart garment is placed on the person so that the smart garment covers at least a portion of the person's body. A plurality of electrodes is attached to the smart garment. At least one sensor is attached to the smart garment. A control module is attached to the smart garment. The plurality of electrodes is operably connected to the control module. The at least one sensor is operably connected to the control module. The person's muscles are stimulated with the plurality of electrodes. Physiological data is collected with the at least one sensor. Data is transferred from the control module to the plurality of electrodes relating to a stimulation protocol. The physiological data is transferred from the at least one sensor to the control module.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 is a front view of a smart garment for use in conjunction with an embodiment of a human body enhanced training system.

FIG. 2 is a back view of the smart garment.

FIG. 3 is a front view of a control module for use in conjunction with the human body enhanced training system.

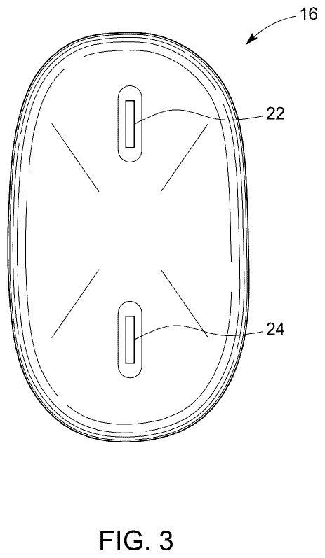

FIG. 4 is a screenshot of a mobile application that is used in conjunction with an embodiment of the human body enhanced training system.

FIG. 5 is another screenshot of a mobile application that illustrates a person's heart rate.

### DETAILED DESCRIPTION OF THE INVENTION

An embodiment of the invention is directed to a human body enhanced training system. The human body enhanced training system may be generally viewed as falling within the category of fitness wearables. One aspect of the human body enhanced training system is electrical muscle stimulation that facilitates stimulating a variety of muscle groups on a human body at the same time while receiving real-time feedback.

The invention is focused in two areas: analytics and muscle recruitment/activation/stimulation. The numerous sensors located all over the person's body enables the system to collect a variety of physiological data such as heart rate, body temperature, respiration rate, hydration, GPS, accelerometer, EKG, X-Y axis position, lactic acid, camera. The system utilizes these measurements to assess overall workout intensity and recovery, which differentiates this invention from the prior art.

Electrical muscle stimulation recruits muscle fibers causing them to contract as opposed to just stimulating nerves using transcutaneous electrical nerve stimulation. Electrical muscle stimulation activates muscles in away that a person cannot do by yourself Many exercises activate slow or fast twitch fibers or slow then fast twitch fibers. Electrical muscle stimulation fires both slow and fast twitch fibers at the same time. Additionally, the firing of the muscles using electrical muscle stimulation is considerably faster than a person can do it naturally. The electrical muscle stimulation thereby enables a person to have a higher intensity workout in a shorter period of time as compared to exercising without the electrical muscle stimulation. Furthermore, whole body electrical muscle stimulation using the full-body garment discussed herein delivers the muscle stimulation equally and simultaneously. through such a process, the invention corrects imbalances that are typically found in the prior art while also preventing injury. The invention also enables delivering different frequencies of muscle stimulation such that the workout can be tailored to a person's particular goals such as strength, endurance, fat-burning and recovery.

The invention applies an electrical current to electrodes on a person's skin for muscle conditioning by temporarily affecting the stimulated muscle's contractile properties, force output and/or fatigue resistance. However, the invention is not intended for use by patients with medical conditions.

A significant advantage of the human body enhanced training system as compared to the prior art fitness wearables is the offers a combination of improved fitness, reduced workout time and optimized performance. In particular, the human body enhanced training system enables the person to reduce the workout time while obtaining better, enhanced physiological results than is possible using a single muscle group electrical stimulation system.

The human body enhanced training system include a smart garment that provides a scalable platform for electrical muscle stimulation that simultaneously collects physiological analytics that may be used to evaluate and optimize the persons workout. Because of the features of the human body enhanced training system, a person can achieve a full body workout in as little as 20 minutes that activates more than 90% of the muscle fibers in the person's body.

In addition to tracking the physiological aspects during the persons workout, it is possible to track the person's recovery after the workout has ended. Using such data, it is possible for the person to structure the workouts with an optimal spacing such that the muscles have had sufficient time to recover and thereby avoid the potential damage to the muscles by working out too soon after a prior workout.

The human body enhanced training system may be used by a variety of different groups of athletes. One such group of athletes that can definitely benefit from the human body enhanced training system are the elite athletes such as found in professional sports leagues and/or the Olympics. The human body enhanced training system may also provide benefits to recreational athletes who are working out at a health or fitness club. These benefits can be achieved through personal training sessions and/or group fitness sessions.

In addition to the recreational athlete benefiting from the use of the human body enhanced training system, the fitness center can also benefit by enhancing member satisfaction and thereby increasing member retention in addition to receiving an additional revenue stream from the use of the human body enhanced training system.

The human body enhanced training system generally includes a smart garment 12 that is worn by a person who is using the human body enhanced training system. The smart garment 12 covers at least a portion of the person's body. In certain embodiments, the smart garment 12 extends over the larger muscles in the person's torso, arms and/or legs.

While it is illustrated that the smart garment 12 is shaped to substantially cover the person's arm and legs, it is possible for the smart garment 12 to be configured to only cover a portion of the person's arms and legs similar to a short-sleeve shirt or shorts.

The smart garment 12 may be configured to fit male bodies or female bodies. While the smart garment 12 is illustrated as being a single piece, it is possible for the smart garment 12 to be configured with an upper torso portion that is separate from a lower body portion. Such a configuration enables only one of the portions to be worn if the person does not want a full body workout.

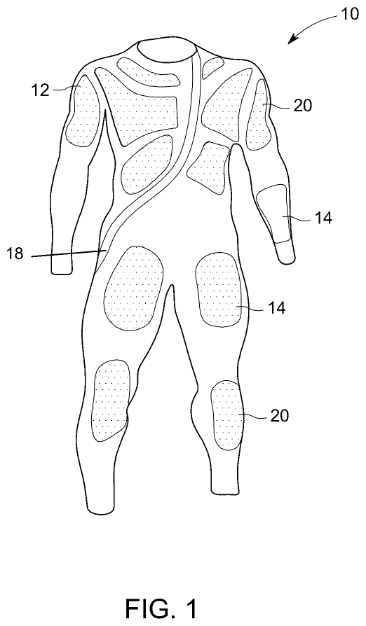

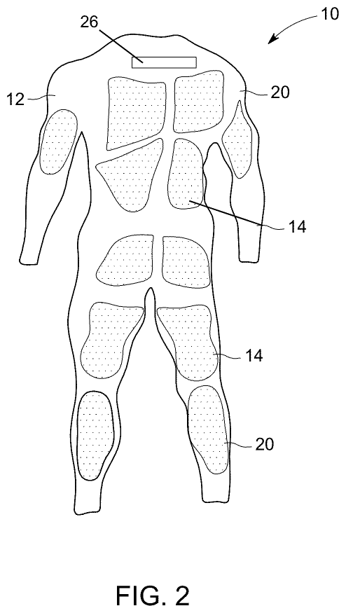

The smart garment 12 illustrated in FIGS. 1 and 2 is configured to substantially cover the front and back surfaces of the person's torso, substantially cover both of the person's arms to proximate the person's hands and substantially cover both of the person's legs to proximate the person's feet.

The smart garment 12 may be fabricated from a stretchy material such that when the smart garment 12 is worn, the smart garment 12 is substantially adjacent to all of the person's skin that is covered by the smart garment 12. A person of skill in the art will appreciate that a variety of materials may be used to fabricate the smart garment 12 having the preceding characteristics. While it is illustrated that the smart garment 12 is fabricated from a continuous sheet of fabric, it is possible for other configurations to be used. Because the smart garment 12 is intended to be worn while a person is working out or performing other strenuous activities, the smart garment 12 may be configured to be washable using a conventional washing machine. In situations where the smart garment 12 is intended to be worn by a firefighter, at least a portion of the smart garment 12 may be fabricated from a flame-retardant material such as NOMEX®.

Depending on the location at which the human body enhanced training system is used, the smart garment 12 may include additional features such as providing heating to the person wearing the smart garment 12. Alternatively or additionally, the smart garment 12 may be capable of providing cooling to the person wearing the smart garment 12.

The heating and/or cooling may be used in conjunction with providing the enhanced training to the person wearing the smart garment 12. For example, the heating and/or cooling may be selectively provided to enhance the ability of the person to recover from a workout and/or to recover from an injury.

The smart garment 12 may include at least one closure mechanism (not shown) that enhances the ability of the person wearing the smart garment 12 to put the smart garment 12 onto the person's body and to remove the smart garment 12 from the person's body. An example of one suitable closure mechanism is a zipper 18.

The human body enhanced training system also includes a plurality of electrodes 14. The electrodes 14 are positioned over at least a portion of the major muscle groups in the person's body. In certain embodiments, at least one of the electrodes 14 is positioned over each of the major muscle groups.

As is described in more detail below, the human body enhanced training system may be configured to selectively use only a portion of the electrodes 14 at a given time and to control the stimulation intensity that is provided by the electrodes 14.

The attachment of the electrodes 14 to the smart garment 12 should enable the electrodes 14 to each remain substantially stationary when the smart garment 12 is placed on the person's body. The electrodes 14 may be removably attached to the smart garment 12 so that the electrodes 14 may be separated from the smart garment 12 such as when it is desired to clean the smart garment 12.

A person of skill in the art will appreciate that a variety of techniques may be used to removably attach the electrodes 14 to the smart garment 12. An example of one suitable technique for attaching the electrodes 14 to the smart garment 12 is a hook and latch fastener that is available under the designation Velcro.

Each of the electrodes 14 is operably attached to a control unit 16. An example of a suitable technique for operably attaching the electrodes 14 to the control unit 16 is a wire. Alternatively, this possible for the electrodes 14 to be wirelessly connected to the control unit 16.

If the electrodes 14 are wirelessly connected to the control unit 16, the electrodes 14 may be provided with a power source (not shown) that is capable of not only providing the stimulation to the person's muscles but also to power communication between the electrode 14 and the control unit 16. In one such configuration, the power source is a battery. The power source may be rechargeable such as from the movement of the person who is wearing the smart garment 12 to which the electrodes 14 are attached. Alternatively or additionally, the power source may be rechargeable such as by connection to an electrical power source.

The human body enhanced training system may also include at least one sensor 20 that is attached to the smart garment 12. The at least one sensor 20 may be integrated with the electrode 14. Alternatively, the at least one sensor 20 may be separate from the electrode 14.

The at least one sensor 20 may have a variety of sensing capabilities. In one configuration, each sensor 20 may have multiple functional sensing capabilities. In another embodiment, each sensor 20 has only one function sensing capability and different sensors are used to provide different sensing capabilities.

Similar to the electrodes 14, the at least one sensor 20 may be positioned in different locations of the smart garment 12. For example, the sensor 20 that measures a person's heart rate maybe positioned in the smart garment 12 over at location at which it is efficient to sense the person's heart rate.

Similarly, the at least one sensor 20 that measures perspiration may be positioned in the smart garment 12 at a location where the person wearing the smart garment 12 is likely to perspire. Examples of additional and/or alternative functionality for the at least one sensor 20 include body temperature, respiration rate, body composition, muscle activation, calories burned, hydration, lactic acid, GPS, accelerometer, steps and sleep.

The attachment of the sensors 20 to the smart garment 12 should enable the sensors 20 to each remain substantially stationary when the smart garment 12 is placed on the person's body. The sensors 20 may be removably attached to the smart garment 12 so that the sensors 20 may be separated from the smart garment 12 such as when it is desired to clean the smart garment 12.

A person of skill in the art will appreciate that a variety of techniques may be used to removably attach the sensors 20 to the smart garment 12. An example of one suitable technique for attaching the sensors 20 to the smart garment 12 is a hook and latch fastener that is available under the designation Velcro.

Each of the sensors 20 is operably attached to a control unit 16. An example of a suitable technique for operably attaching the sensors 20 to the control unit 16 is a wire. Alternatively, this possible for the sensors 20 to be wirelessly connected to the control unit 16.

If the sensors 20 are wirelessly connected to the control unit 16, the sensors 20 may be provided with a power source (not shown) that is capable of not only collecting the data from the person but also to power communication between the sensors 20 and the control unit 16.

The communication between the control unit 16 and the sensors 20 may utilize a robust security protocol and/or data encryption to ensure that the data is not accessed by unauthorized persons. A person of skill in the art will appreciate that there are a variety of suitable security protocols and data encryption techniques.

In one such configuration, the power source is a battery. The power source may be rechargeable such as from the movement of the person who is wearing the smart garment 12 to which the sensors 20 are attached. Alternatively or additionally, the power source may be rechargeable such as by connection to an electrical power source.

The control unit 16 receives data from the electrodes 14 and the sensors 20. It is also possible for the control unit 16 to include functionality that is similar to the electrodes 14 and/or the sensors 20.

The control unit 16 may include memory that stores data received from the sensors 20 until the data is transferred such as to a mobile phone or tablet computer. A person of skill in the art will appreciate that the memory may have a variety of configuration using the concepts of the invention.

The control unit 16 transfers this data such as to an application on a mobile phone or through the Internet to a provider that operates a website. The mobile application or website serves as a command and control center. Such a process enables the person using the human body enhanced training system to view data associated with the workouts as well as to receive suggestions on maximizing the effectiveness of using the electrical muscle stimulation system 10.

Such a process could be viewed as a post workout debrief. The human body enhanced training system may be configured to directly send this information to the person who was wearing the smart garment 12 after the workout is completed. Alternatively or additionally, the human body enhanced training system may send the data to a coach or trainer who evaluates the data and then reviews with the person who was wearing the smart garment 12.

In certain embodiments, the data can be transferred between the control unit 16 and the other device using a variety of wireless techniques such as Bluetooth, Wi-Fi and a cellular network. Alternatively, the control unit 16 may transfer the data through a physical connection such as a cable that extends between the control unit 16 and a mobile phone or a computer.

The control unit 16 may have a power source such as a battery that is mounted therein. The power source thereby provides power to the control unit 16 to collect data from the electrodes and/or the sensors 20 as well as to communicate with the associated mobile phone, computer or other data collection system.

The communication between the control unit 16 and the mobile phone may utilize a robust security protocol and/or data encryption to ensure that the data is not accessed by unauthorized persons. A person of skill in the art will appreciate that there are a variety of suitable security protocols and data encryption techniques.

The power source may be rechargeable such as from the movement of the person who is wearing the smart garment 12 to which the control unit 16 is attached. Alternatively or additionally, the power source may be rechargeable such as by connection to an electrical power source.

The control unit 16 may include at least one button 22 that may be used for turning the control unit 16 on or off. The at least one button 22 may also be used in wirelessly linking control unit 16 to the mobile phone, computer or other data collection system.

The control unit 16 may also include at least one light 24 that may be used for visualizing an operational status of the control unit 16. In certain embodiments, the at least one light 24 may illuminate to indicate that the control unit 16 is turned on. The at least one light 24 may also indicate a power level of the power source and/or a link status of the control unit 16 and the mobile phone, computer or other data collection system.

The control unit 16 maybe removably attached to the smart garment 12 in a location that minimizes the presence of the control unit 16 interfering with the fitness activities that are being done by the person wearing the smart garment 12. However, the control unit 16 should be removably attached to the smart garment 12 such that the control unit 16 may be separated from the smart garment 12 such as when it is desired to clean the smart garment 12.

One technique for attaching the control unit 16 to the smart garment 12 is by placing the control unit 16 into a pouch 26 on the smart garment 12. The pouch 26 may be positioned on the smart garment 12 so that the presence of the control unit 16 minimizes interference with the activities being performed by the person wearing the smart garment 12. Depending on factors such as the size of the control unit 16, a closure mechanism (not shown) may be provided on the pouch 26.

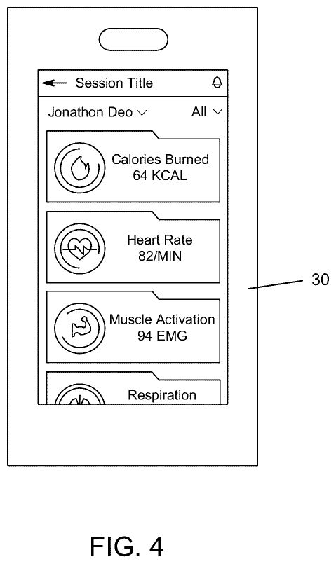

In one embodiment, the human body enhanced training system may include an application that is installed on a mobile phone 30, such as illustrated in FIG. 4. The application may be used in conjunction with controlling and/or configuring various operation aspects of the human body enhanced training system. The application may also be used for analyzing data that is transferred to the mobile phone from the electrodes 14 and/or the sensors 20 using the control unit 16.

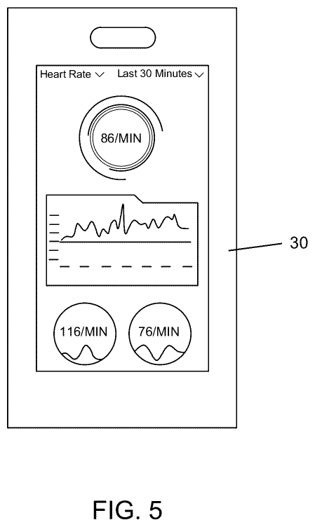

In an initial screen of the mobile application, which is illustrated in FIG. 4, there may be a listing of the various conditions that are being monitored by the system. When one of these conditions is selected such as by clicking on the screen, more detailed information is displayed such as in FIG. 5, which provides details about the person's heart rate.

In operation, a person selects a style of the smart garment 12 based upon the muscle groups with which the person desires to utilize the electrical muscle stimulation system 10. In one such embodiment, the smart garment 12 is configured like illustrated in FIGS. 1 and 2 so that the smart garment 12 substantially covers the front and back of the person's torso, substantially covers the person's arms to proximate the person's hands and substantially covers the person's legs to proximate the person's feet.

As described above, the smart garment 12 is selected with a size based on the size of the person's body such that the smart garment 12 is positioned along the person skin in the portions of the person's body that is covered by the smart garment 12.

Prior to placing the smart garment 12 on the person's body, the person attaches electrodes 14 to the smart garment 12 in each of the desired locations at which the person desires to receive the electrical muscle stimulation. Additionally, the person attaches the sensors 20 to the smart garment at each of the locations based on the type of sensor 20 that is being used. Alternatively, the electrodes 14 and the sensors 20 may be attached to the smart garment 12 after the smart garment 12 is placed on the person's body.

Either before, during or after the person is doing the desired exercises, electrical muscle stimulation system is activated such that the electrodes 14 deliver electrical pulses to the person's muscles, which cause stimulation of the person's muscles. The intensity of the stimulation can be selected based upon a variety of factors to provide the person with a desired level of electrical muscle stimulation.

The electrical muscle stimulation system 10 may be used in a variety of configurations to produce different results. examples of these protocols include strength, endurance, fat burning and yoga recovery.

The use of the electrical muscle stimulation system 10 thereby causes a greater percentage of the persons muscle fibers to be engaged as compared to exercising without the electrical muscle stimulation system 10. In certain configurations, greater than about 90% of the muscle fibers over which the electrodes 14 are used are caused to be stimulated by the electrical muscle stimulation system 10.

The electrical muscle stimulation system 10 increases explosive strength, avoids training fatigue, reduces muscle enjoyment damage and provides faster recovery. The person using the electrical muscle stimulation system 10 thereby is able to achieve superior results as compared to similar exercises done without the use of the electrical muscle stimulation system 10. In many circumstances, the electrical muscle stimulation system 10 provides a reduction in the duration of the exercise needed to achieve these results as compared to performing the exercises without the electrical muscle stimulation system 10.

Either before, during or after the person is doing the desired exercises, the sensors 20 may be used to collect data that is associated with the functionality of the sensors. These sensors 20 may be activated either together with or separate from the electrodes 14.

While at least one of the electrodes 14 and the sensors 20 are activated, data associated with such activation may be transferred to the control unit 16. This data may be retained in the control unit 16 while the smart garment 12 is being worn and then may be transferred to an external data analysis system after the smart garment 12 is removed. Alternatively, the data that is received in the control unit 16 may be transferred to the external data analysis system while the smart garment 12 is being worn and or while the person is performing the exercises.

As mentioned above the closed loop, physiological data that can be acquired by the electrical muscle stimulation system 10 includes heart rate, body temperature, respiration rate, body composition, muscle activation, calories burned, hydration, lactic acid, GPS, accelerometer, steps and sleep.

Through the combination of the GPS data and the other data that is received by the sensors 20, it is possible to evaluate the performance of the person using the electrical muscle stimulation system 10 in different locations such as going up an incline, climbing stairs or running on a flat ground surface.

Through the repeated use of the electrical muscle stimulation system 10, it is possible for periodic fitness test to be conducted for the person who is wearing the smart garment 12. The periodic fitness test can assess readiness, performance and recovery. By comparing the results of different tests performed at different times, it may be possible to identify potential issues before the person wearing the electrical muscle stimulation system 10 feels any pain or other symptoms. The readiness assessment thereby assists the person wearing the electrical muscle stimulation system 10 to avoid injuries.

Depending on the situation in which it is intended to use the invention, one of the smart garments 12 may be associated with a single mobile device. Such a configuration facilitates a person monitoring his/her workout. As is described in more detail above, the invention thereby enables the person to receive real-time feedback either during a workout or during a recovery period after a workout.

Alternatively, multiple smart garments 12 may be associated with a single mobile device. Such a configuration may be used at a fitness club that enables one trainer to simultaneously oversee multiple people who are working out. This configuration may also be used such as by a sports team so that multiple athletes can simultaneously workout. When the invention is used with multiple persons, the display on the mobile device may include a grid so that the person monitoring the workout can simultaneously see the data from each of the persons being monitored or from at least a portion of the persons who are being monitored.

An example of one real life application of the periodic readiness testing is evaluating firefighters for heart abnormalities such that the firefighters do not experience significant medical conditions such as a heart attack when responding to a fire. A leading cause of firefighter deaths is from heart attacks and exertion while fighting structural fires.

The results of these fitness tests can be used to track the progress such as when recovering from an injury or a surgery. The data from the electrical muscle stimulation system 10 they enable changes to be made in the stimulation intensity, stimulation duration or stimulation protocol while the person is actually performing the exercise. Through such use, there can be corrections in the exercise protocol in real time and through such corrections, the person is more likely to recover quicker while also avoiding injuries that could derail the recovery process.

In still another embodiment of the invention, the electrical muscle stimulation system 10 is used to provide real time information to an incident commander who is associated with firefighters. The electrical muscle stimulation system 10 thereby assist the incident commander in making critical, split second decision to improve the safety of firefighters as well as the people the firefighters are serving.

Alternatively, the electrical muscle stimulation system 10 may be used by coaches of professional athletes to monitor the performance of these athletes during athletic competitions and through such monitoring, the coaches will have greater detail on when the athlete is experiencing exhaustion or other muscle issues such that the athlete should be substituted out of the game. Where it is not possible for the coach to monitor the performance of the athlete during an actual game, the monitoring of the athlete using the electrical muscle stimulation system 10 may be used during practice sessions that are conducted under game conditions. The results of the monitoring of the athlete's performance using the

In the preceding detailed description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The preceding detailed description, therefore, is not to be taken in a limiting sense, and the scope of the present invention is defined by the appended claims.

It is contemplated that features disclosed in this application, as well as those described in the above applications incorporated by reference, can be mixed and matched to suit particular circumstances. Various other modifications and changes will be apparent to those of ordinary skill.

1. A human body enhanced training system comprising:
a smart garment that is suitable for being worn by a person, wherein when the smart garment is worn by the person, the smart garment covers at least a portion of the person's body;
a control module mounted with respect to the smart garment;
a plurality of electrodes operably connected to the control module, wherein the plurality of electrodes is capable of stimulating the person's muscles; and
at least one sensor operably connected to the control module, wherein the at least one sensor is capable of collecting physiological data

2. The human body enhanced training system of claim 1, wherein when the smart garment is worn, the smart garment at least partially covers larger muscle groups in the person's torso, arms and legs.

3. The human body enhanced training system of claim 1, wherein the smart garment is fabricated from a stretchy material such that when the smart garment is worn, the smart garment is substantially adjacent to the person's skin that is covered by the smart garment.

4. The human body enhanced training system of claim 1, wherein the plurality of electrodes and the at least one sensor are removably attached to the smart garment.

5. The human body enhanced training system of claim 1, wherein the plurality of electrodes and the at least one sensor wirelessly communicate with the control module.

6. The human body enhanced training system of claim 1, wherein the at least one sensor is capable of sensing at least one of heart rate, body temperature, body composition, respiration rate, muscle activation, calories burned, hydration, lactic acid concentration, GPS location, acceleration, steps and sleep.

7. The human body enhanced training system of claim 1, wherein the smart garment is capable of providing heating and/or cooling to the person's body.

8. A method of training comprising:
providing a smart garment that is suitable for being worn by a person, wherein the person has a body and muscles;
placing the smart garment on the person so that the smart garment covers at least a portion of the person's body;
attaching a plurality of electrodes to the smart garment;
attaching at least one sensor to the smart garment;
attaching a control module to the smart garment;
operably connecting the plurality of electrodes to the control module;
operably connecting the at least one sensor to the control module;
stimulating the person's muscles with the plurality of electrodes;
collecting physiological data with the at least one sensor;
transferring data from the control module to the plurality of electrodes relating to a stimulation protocol; and
transferring the physiological data from the at least one sensor to the control module.

9. The method of claim 8, and further comprising:
selectively attaching the electrodes to the smart garment based upon the muscle desired to be stimulated; and
selectively attaching the at least one sensor to the smart garment based upon what physiological data the at least one sensor is intended to sense.

10. The method of claim 8, wherein the stimulation activates greater than about 90 percent of muscle in the person's body in a workout session of less than about 20 minutes.

11. The method of claim 8, wherein the collected physiological data comprises at least one of heart rate, body temperature, body composition, respiration rate, muscle activation, calories burned, hydration, lactic acid concentration, GPS location, acceleration, steps and sleep.

12. The method of claim 8, and further comprising
performing a workout that exercises the muscles;
tracking recovery of the person after the workout with the at least one sensor; and
based upon the person's recovery, scheduling additional workouts.

13. The method of claim 8, wherein the stimulating of the person's muscles and the collecting the physiological data are done while the person is conducting an activity and wherein the method further comprises modifying the activity based the collected physiological data while the activity is being performed.

14. The method of claim 13, wherein the activity is exercise performed at a fitness center or a sports competition

15. The method of claim 13, wherein the activity is fighting a fire.

16. The method of claim 8, wherein the smart garment at least partially covers larger muscle groups in the person's torso, arms and legs.

17. The method of claim 8, and further comprising fabricating the smart garment from a stretchy material such that when the smart garment is worn, the smart garment is substantially adjacent to the person's skin that is covered by the smart garment.

18. The method of claim 8, and further comprising removably attaching the plurality of electrodes and the at least one sensor to the smart garment.

19. The method of claim 8, and further comprising wirelessly connecting the plurality of electrodes and the at least one sensor to the control module.

20. The method of claim 8, and further comprising heating and/or cooling at least a portion of the person's body with the smart garment.
